(19) United States
(12) Patent Application Publication
(10) Pub. No.: US 2017/0007640 A1
(43) Pub. Date: Jan. 12, 2017

(54) METHOD AND SYSTEM FOR EXTRACTING AND USING MOOMIYO COMPOSITIONS

(71) Applicants: Sergei Smirnov, Manalapan, NJ (US); Elena Smirnov, Manalapan, NJ (US)

(72) Inventors: Sergei Smirnov, Manalapan, NJ (US); Elena Smirnov, Manalapan, NJ (US)

(21) Appl. No.: 14/792,826

(22) Filed: Jul. 7, 2015

(51) Int. Cl. A61K 33/00; B01D 11/02

(52) U.S. Cl. CPC A61K 33/00; B01D 11/0288

(57) ABSTRACT

A method and system for extraction of moomiyo from a multi-component, naturally-occurring mineral substance generally found in Asian mountain ranges at elevations approximately between 1000 and 5000 meters. Bio-active compounds are extracted from said mineral substance for the medicinal applications including at least one of joint pain relief, skin care, anti-inflammation, bruise removal, decrease high blood cholesterol and high triglycerides levels, promotion of mental and physical energy, regulation of blood sugar levels, stimulation of the immune system, speeding of the recovery from sickness and wound healing therapies.

### CROSS-REFERENCE TO RELATED APPLICATIONS

Not applicable.

### RELATED CO-PENDING U.S. PATENT APPLICATIONS

Not applicable.

### FEDERALLY SPONSORED RESEARCH OR DEVELOPMENT

Not applicable.

### REFERENCE TO SEQUENCE LISTING, A TABLE, OR A COMPUTER LISTING APPENDIX

Not applicable.

### COPYRIGHT NOTICE

A portion of the disclosure of this patent document contains material that is subject to copyright protection. The copyright owner has no objection to the facsimile reproduction by anyone of the patent document or patent disclosure as it appears in the Patent and Trademark Office, patent file or records, but otherwise reserves all copyright rights whatsoever.

### FIELD OF THE INVENTION

One or more embodiments of the invention generally relate to moomiyo compositions and mixtures. More particularly, the invention relates to the extraction of moomiyo containing bioactive compounds capable of improving regenerative processes.

### BACKGROUND OF THE INVENTION

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

Moomiyo (also known as shilajit, silajit, salajeet or mumijo, and momia) is a wax-like exudation from cracks in mountainous rocks generally found in Asian mountain ranges at elevations between 1000 and 5000 meters. It is a multi-component, naturally-occurring, mineral substance appearing yellowish-brown to pitch-black depending on composition. It is believed that moomiyo may contain over 85 minerals which are believed to promote healing processes and have been used for medical purposes.

The following is an example of a specific aspect in the prior art that, while expected to be helpful to further educate the reader as to additional aspects of the prior art, is not to be construed as limited the present invention, or any embodiments thereof, to anything stated or implied therein or inferred thereupon. By way of educational background, another aspect of the prior art generally useful to be aware of is that extraction procedures for shilajit may include dissolving in water as a solvent and then using a hot methanol.

In view of the foregoing, it is clear that traditional techniques of moomiyo extraction are not perfect and leave room for more optimal approaches.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1 illustrates a method for moomiyo extraction, in accordance with an embodiment of the present invention;

FIG. 2 depicts the effect of the ethanol extracted moomiyo on wound healing in a dose-dependent manner, in accordance with an embodiment of the present invention;

FIG. 3 depicts the effect of different moomiyo extracts on wound healing, in accordance with an embodiment of the present invention;

FIG. 4 depicts the effect of alcohol extracted moomiyo on granulation tissue formation, in accordance with an embodiment of the present invention;

FIG. 5 depicts the effect of moomiyo cream on wound healing, in accordance with an embodiment of the present invention; and

FIG. 6 is a histology analysis of wounded skin, in accordance with an embodiment of the present invention.

Unless otherwise indicated illustrations in the figures are not necessarily drawn to scale.

### DETAILED DESCRIPTION OF SOME EMBODIMENTS

The present invention is best understood by reference to the detailed figures and description set forth herein.

Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

It is to be further understood that the present invention is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

All words of approximation as used in the present disclosure and claims should be construed to mean “approximate,” rather than “perfect,” and may accordingly be employed as a meaningful modifier to any other word, specified parameter, quantity, quality, or concept. Words of approximation, include, yet are not limited to terms such as “substantial”, “nearly”, “almost”, “about”, “generally”, “largely”, “essentially”, “closely approximate”, etc.

As will be established in some detail below, it is well settle law, as early as 1939, that words of approximation are not indefinite in the claims even when such limits are not defined or specified in the specification.

For example, see Ex parte Mallory, 52 USPQ 297, 297 (Pat. Off. Bd. App. 1941) where the court said “The examiner has held that most of the claims are inaccurate because apparently the laminar film will not be entirely eliminated. The claims specify that the film is “substantially” eliminated and for the intended purpose, it is believed that the slight portion of the film which may remain is negligible. We are of the view, therefore, that the claims may be regarded as sufficiently accurate.”

Note that claims need only “reasonably apprise those skilled in the art” as to their scope to satisfy the definiteness requirement. See Energy Absorption Sys., Inc. v. Roadway Safety Servs., Inc., Civ. App. 96-1264, slip op. at 10 (Fed. Cir. Jul. 3, 1997) (unpublished) Hybridtech v. Monoclonal Antibodies, Inc., 802 F.2d 1367, 1385, 231 USPQ 81, 94 (Fed. Cir. 1986), cert. denied, 480 U.S. 947 (1987). In addition, the use of modifiers in the claim, like “generally” and “substantial,” does not by itself render the claims indefinite. See Seattle Box Co. v. Industrial Crating & Packing, Inc., 731 F.2d 818, 828-29, 221 USPQ 568, 575-76 (Fed. Cir. 1984).

Moreover, the ordinary and customary meaning of terms like “substantially” includes “reasonably close to: nearly, almost, about”, connoting a term of approximation. See In re Frye, Appeal No. 2009-006013, 94 USPQ2d 1072, 1077, 2010 WL 889747 (B.P.A.I. 2010) Depending on its usage, the word “substantially” can denote either language of approximation or language of magnitude. Deering Precision Instruments, L.L.C. v. Vector Distribution Sys., Inc., 347 F.3d 1314, 1323 (Fed. Cir. 2003) (recognizing the “dual ordinary meaning of th[e] term [“substantially”] as connoting a term of approximation or a term of magnitude”). Here, when referring to the “substantially halfway” limitation, the Specification uses the word “approximately” as a substitute for the word “substantially” (Fact 4). (Fact 4). The ordinary meaning of “substantially halfway” is thus reasonably close to or nearly at the midpoint between the forwardmost point of the upper or outsole and the rearwardmost point of the upper or outsole.

Similarly, the term ‘substantially’ is well recognize in case law to have the dual ordinary meaning of connoting a term of approximation or a term of magnitude. See Dana Corp. v. American Axle & Manufacturing, Inc., Civ. App. 04-1116, 2004 U.S. App. LEXIS 18265, *13-14 (Fed. Cir. Aug. 27, 2004) (unpublished). The term “substantially” is commonly used by claim drafters to indicate approximation. See Cordis Corp. v. Medtronic AVE Inc., 339 F.3d 1352, 1360 (Fed. Cir. 2003) (“The patents do not set out any numerical standard by which to determine whether the thickness of the wall surface is ‘substantially uniform.’ The term ‘substantially,’ as used in this context, denotes approximation. Thus, the walls must be of largely or approximately uniform thickness.”); see also Deering Precision Instruments, LLC v. Vector Distribution Sys., Inc., 347 F.3d 1314, 1322 (Fed. Cir. 2003); Epcon Gas Sys., Inc. v. Bauer Compressors, Inc., 279 F.3d 1022, 1031 (Fed. Cir. 2002). We find that the term “substantially” was used in just such a manner in the claims of the patents-in-suit: “substantially uniform wall thickness” denotes a wall thickness with approximate uniformity.

It should also be noted that such words of approximation as contemplated in the foregoing clearly limits the scope of claims such as saying ‘generally parallel’ such that the adverb ‘generally’ does not broaden the meaning of parallel. Accordingly, it is well settled that such words of approximation as contemplated in the foregoing (e.g., like the phrase ‘generally parallel’) envisions some amount of deviation from perfection (e.g., not exactly parallel), and that such words of approximation as contemplated in the foregoing are descriptive terms commonly used in patent claims to avoid a strict numerical boundary to the specified parameter. To the extent that the plain language of the claims relying on such words of approximation as contemplated in the foregoing are clear and uncontradicted by anything in the written description herein or the figures thereof, it is improper to rely upon the present written description, the figures, or the prosecution history to add limitations to any of the claim of the present invention with respect to such words of approximation as contemplated in the foregoing. That is, under such circumstances, relying on the written description and prosecution history to reject the ordinary and customary meanings of the words themselves is impermissible. See, for example, Liquid Dynamics Corp. v. Vaughan Co., 355 F.3d 1361, 69 USPQ2d 1595, 1600-01 (Fed. Cir. 2004). The plain language of phrase 2 requires a “substantial helical flow.” The term “substantial” is a meaningful modifier implying “approximate,” rather than “perfect.” In Cordis Corp. v. Medtronic AVE, Inc., 339 F.3d 1352, 1361 (Fed. Cir. 2003), the district court imposed a precise numeric constraint on the term “substantially uniform thickness.” We noted that the proper interpretation of this term was “of largely or approximately uniform thickness” unless something in the prosecution history imposed the “clear and unmistakable disclaimer” needed for narrowing beyond this simple-language interpretation. Id. In Anchor Wall Systems v. Rockwood Retaining Walls, Inc., 340 F.3d 1298, 1311 (Fed. Cir. 2003)” Id. at 1311. Similarly, the plain language of claim 1 requires neither a perfectly helical flow nor a flow that returns precisely to the center after one rotation (a limitation that arises only as a logical consequence of requiring a perfectly helical flow).

The reader should appreciate that case law generally recognizes a dual ordinary meaning of such words of approximation, as contemplated in the foregoing, as connoting a term of approximation or a term of magnitude; e.g., see Deering Precision Instruments, L.L.C. v. Vector Distrib. Sys., Inc., 347 F.3d 1314, 68 USPQ2d 1716, 1721 (Fed. Cir. 2003), cert. denied, 124 S. Ct. 1426 (2004) where the court was asked to construe the meaning of the term “substantially” in a patent claim. Also see Epcon, 279 F.3d at 1031 (“The phrase ‘substantially constant’ denotes language of approximation, while the phrase ‘substantially below’ signifies language of magnitude, i.e., not insubstantial.”). Also, see, e.g., Epcon Gas Sys., Inc. v. Bauer Compressors, Inc., 279 F.3d 1022 (Fed. Cir. 2002) (construing the terms “substantially constant” and “substantially below”); Zodiac Pool Care, Inc. v. Hoffinger Indus., Inc., 206 F.3d 1408 (Fed. Cir. 2000) (construing the term “substantially inward”); York Prods., Inc. v. Cent. Tractor Farm & Family Ctr., 99 F.3d 1568 (Fed. Cir. 1996) (construing the term “substantially the entire height thereof”); Tex. Instruments Inc. v. Cypress Semiconductor Corp., 90 F.3d 1558 (Fed. Cir. 1996) (construing the term “substantially in the common plane”). In conducting their analysis, the court instructed to begin with the ordinary meaning of the claim terms to one of ordinary skill in the art. Prima Tek, 318 F.3d at 1148. Reference to dictionaries and our cases indicates that the term “substantially” has numerous ordinary meanings. As the district court stated, “substantially” can mean “significantly” or “considerably.” The term “substantially” can also mean “largely” or “essentially.” Webster's New 20th Century Dictionary 1817 (1983).

Words of approximation, as contemplated in the foregoing, may also be used in phrases establishing approximate ranges or limits, where the end points are inclusive and approximate, not perfect; e.g., see AK Steel Corp. v. Sollac, 344 F.3d 1234, 68 USPQ2d 1280, 1285 (Fed. Cir. 2003) where it where the court said [W]e conclude that the ordinary meaning of the phrase “up to about 10%” includes the “about 10%” endpoint. As pointed out by AK Steel, when an object of the preposition “up to” is nonnumeric, the most natural meaning is to exclude the object (e.g., painting the wall up to the door). On the other hand, as pointed out by Sollac, when the object is a numerical limit, the normal meaning is to include that upper numerical limit (e.g., counting up to ten, seating capacity for up to seven passengers). Because we have here a numerical limit—“about 10%”—the ordinary meaning is that that endpoint is included.

In the present specification and claims, a goal of employment of such words of approximation, as contemplated in the foregoing, is to avoid a strict numerical boundary to the modified specified parameter, as sanctioned by Pall Corp. v. Micron Separations, Inc., 66 F.3d 1211, 1217, 36 USPQ2d 1225, 1229 (Fed. Cir. 1995) where it states “It is well established that when the term “substantially” serves reasonably to describe the subject matter so that its scope would be understood by persons in the field of the invention, and to distinguish the claimed subject matter from the prior art, it is not indefinite.” Likewise see Verve LLC v. Crane Cams Inc., 311 F.3d 1116, 65 USPQ2d 1051, 1054 (Fed. Cir. 2002). Expressions such as “substantially” are used in patent documents when warranted by the nature of the invention, in order to accommodate the minor variations that may be appropriate to secure the invention. Such usage may well satisfy the charge to “particularly point out and distinctly claim” the invention, 35 U.S.C. §112, and indeed may be necessary in order to provide the inventor with the benefit of his invention. In Andrew Corp. v. Gabriel Elecs. Inc., 847 F.2d 819, 821-22, 6 USPQ2d 2010, 2013 (Fed. Cir. 1988) the court explained that usages such as “substantially equal” and “closely approximate” may serve to describe the invention with precision appropriate to the technology and without intruding on the prior art. The court again explained in Ecolab Inc. v. Envirochem, Inc., 264 F.3d 1358, 1367, 60 USPQ2d 1173, 1179 (Fed. Cir. 2001) that “like the term ‘about,’ the term ‘substantially’ is a descriptive term commonly used in patent claims to ‘avoid a strict numerical boundary to the specified parameter, see Ecolab Inc. v. Envirochem Inc., 264 F.3d 1358, 60 USPQ2d 1173, 1179 (Fed. Cir. 2001) where the court found that the use of the term “substantially” to modify the term “uniform” does not render this phrase so unclear such that there is no means by which to ascertain the claim scope.

Similarly, other courts have noted that like the term “about,” the term “substantially” is a descriptive term commonly used in patent claims to “avoid a strict numerical boundary to the specified parameter.”; e.g., see Pall Corp. v. Micron Seps., 66 F.3d 1211, 1217, 36 USPQ2d 1225, 1229 (Fed. Cir. 1995); see, e.g., Andrew Corp. v. Gabriel Elecs. Inc., 847 F.2d 819, 821-22, 6 USPQ2d 2010, 2013 (Fed. Cir. 1988) (noting that terms such as “approach each other,” “close to,” “substantially equal,” and “closely approximate” are ubiquitously used in patent claims and that such usages, when serving reasonably to describe the claimed subject matter to those of skill in the field of the invention, and to distinguish the claimed subject matter from the prior art, have been accepted in patent examination and upheld by the courts). In this case, “substantially” avoids the strict 100% nonuniformity boundary.

Indeed, the foregoing sanctioning of such words of approximation, as contemplated in the foregoing, has been established as early as 1939, see Ex parte Mallory, 52 USPQ 297, 297 (Pat. Off. Bd. App. 1941) where, for example, the court said “the claims specify that the film is “substantially” eliminated and for the intended purpose, it is believed that the slight portion of the film which may remain is negligible. We are of the view, therefore, that the claims may be regarded as sufficiently accurate.” Similarly, In re Hutchison, 104 F.2d 829, 42 USPQ 90, 93 (C.C.P.A. 1939) the court said “It is realized that “substantial distance” is a relative and somewhat indefinite term, or phrase, but terms and phrases of this character are not uncommon in patents in cases where, according to the art involved, the meaning can be determined with reasonable clearness.”

Hence, for at least the forgoing reason, Applicants submit that it is improper for any examiner to hold as indefinite any claims of the present patent that employ any words of approximation.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be understood also to refer to functional equivalents of such structures. The present invention will now be described in detail with reference to embodiments thereof as illustrated in the accompanying drawings.

From reading the present disclosure, other variations and modifications will be apparent to persons skilled in the art. Such variations and modifications may involve equivalent and other features which are already known in the art, and which may be used instead of or in addition to features already described herein.

Although Claims have been formulated in this Application to particular combinations of features, it should be understood that the scope of the disclosure of the present invention also includes any novel feature or any novel combination of features disclosed herein either explicitly or implicitly or any generalization thereof, whether or not it relates to the same invention as presently claimed in any Claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable subcombination. The Applicants hereby give notice that new Claims may be formulated to such features and/or combinations of such features during the prosecution of the present Application or of any further Application derived therefrom.

References to “one embodiment,” “an embodiment,” “example embodiment,” “various embodiments,” “some embodiments,” “embodiments of the invention,” etc., may indicate that the embodiment(s) of the invention so described may include a particular feature, structure, or characteristic, but not every possible embodiment of the invention necessarily includes the particular feature, structure, or characteristic. Further, repeated use of the phrase “in one embodiment,” or “in an exemplary embodiment,” “an embodiment,” do not necessarily refer to the same embodiment, although they may. Moreover, any use of phrases like “embodiments” in connection with “the invention” are never meant to characterize that all embodiments of the invention must include the particular feature, structure, or characteristic, and should instead be understood to mean “at least some embodiments of the invention” includes the stated particular feature, structure, or characteristic.

References to “user”, or any similar term, as used herein, may mean a human or non-human user thereof. Moreover, “user”, or any similar term, as used herein, unless expressly stipulated otherwise, is contemplated to mean users at any stage of the usage process, to include, without limitation, direct user(s), intermediate user(s), indirect user(s), and end user(s). The meaning of “user”, or any similar term, as used herein, should not be otherwise inferred or induced by any pattern(s) of description, embodiments, examples, or referenced prior-art that may (or may not) be provided in the present patent.

References to “end user”, or any similar term, as used herein, is generally intended to mean late stage user(s) as opposed to early stage user(s). Hence, it is contemplated that there may be a multiplicity of different types of “end user” near the end stage of the usage process. Where applicable, especially with respect to distribution channels of embodiments of the invention comprising consumed retail products/services thereof (as opposed to sellers/vendors or Original Equipment Manufacturers), examples of an “end user” may include, without limitation, a “consumer”, “buyer”, “customer”, “purchaser”, “shopper”, “enjoyer”, “viewer”, or individual person or non-human thing benefiting in any way, directly or indirectly, from use of or interaction, with some aspect of the present invention.

In some situations, some embodiments of the present invention may provide beneficial usage to more than one stage or type of usage in the foregoing usage process. In such cases where multiple embodiments targeting various stages of the usage process are described, references to “end user”, or any similar term, as used therein, are generally intended to not include the user that is the furthest removed, in the foregoing usage process, from the final user therein of an embodiment of the present invention.

Where applicable, especially with respect to retail distribution channels of embodiments of the invention, intermediate user(s) may include, without limitation, any individual person or non-human thing benefiting in any way, directly or indirectly, from use of, or interaction with, some aspect of the present invention with respect to selling, vending, Original Equipment Manufacturing, marketing, merchandising, distributing, service providing, and the like thereof.

References to “person”, “individual”, “human”, “a party”, “animal”, “creature”, or any similar term, as used herein, even if the context or particular embodiment implies living user, maker, or participant, it should be understood that such characterizations are sole by way of example, and not limitation, in that it is contemplated that any such usage, making, or participation by a living entity in connection with making, using, and/or participating, in any way, with embodiments of the present invention may be substituted by such similar performed by a suitably configured non-living entity, to include, without limitation, automated machines, robots, humanoids, computational systems, information processing systems, artificially intelligent systems, and the like. It is further contemplated that those skilled in the art will readily recognize the practical situations where such living makers, users, and/or participants with embodiments of the present invention may be in whole, or in part, replaced with such non-living makers, users, and/or participants with embodiments of the present invention. Likewise, when those skilled in the art identify such practical situations where such living makers, users, and/or participants with embodiments of the present invention may be in whole, or in part, replaced with such non-living makers, it will be readily apparent in light of the teachings of the present invention how to adapt the described embodiments to be suitable for such non-living makers, users, and/or participants with embodiments of the present invention. Thus, the invention is thus to also cover all such modifications, equivalents, and alternatives falling within the spirit and scope of such adaptations and modifications, at least in part, for such non-living entities.

Headings provided herein are for convenience and are not to be taken as limiting the disclosure in any way.

The enumerated listing of items does not imply that any or all of the items are mutually exclusive, unless expressly specified otherwise.

It is understood that the use of specific component, device and/or parameter names are for example only and not meant to imply any limitations on the invention. The invention may thus be implemented with different nomenclature/terminology utilized to describe the mechanisms/units/structures/components/devices/parameters herein, without limitation. Each term utilized herein is to be given its broadest interpretation given the context in which that term is utilized.

Terminology. The following paragraphs provide definitions and/or context for terms found in this disclosure (including the appended claims):

“Comprising.” This term is open-ended. As used in the appended claims, this term does not foreclose additional structure or steps. Consider a claim that recites: “A memory controller comprising a system cache . . . . ” Such a claim does not foreclose the memory controller from including additional components (e.g., a memory channel unit, a switch).

“Configured To.” Various units, circuits, or other components may be described or claimed as “configured to” perform a task or tasks. In such contexts, “configured to” or “operable for” is used to connote structure by indicating that the mechanisms/units/circuits/components include structure (e.g., circuitry and/or mechanisms) that performs the task or tasks during operation. As such, the mechanisms/unit/circuit/component can be said to be configured to (or be operable) for perform(ing) the task even when the specified mechanisms/unit/circuit/component is not currently operational (e.g., is not on). The mechanisms/units/circuits/components used with the “configured to” or “operable for” language include hardware—for example, mechanisms, structures, electronics, circuits, memory storing program instructions executable to implement the operation, etc. Reciting that a mechanism/unit/circuit/component is “configured to” or “operable for” perform(ing) one or more tasks is expressly intended not to invoke 35 U.S.C. .sctn.112, sixth paragraph, for that mechanism/unit/circuit/component. “Configured to” may also include adapting a manufacturing process to fabricate devices or components that are adapted to implement or perform one or more tasks.

“Based On.” As used herein, this term is used to describe one or more factors that affect a determination. This term does not foreclose additional factors that may affect a determination. That is, a determination may be solely based on those factors or based, at least in part, on those factors. Consider the phrase “determine A based on B.” While B may be a factor that affects the determination of A, such a phrase does not foreclose the determination of A from also being based on C. In other instances, A may be determined based solely on B.

The terms “a”, “an” and “the” mean “one or more”, unless expressly specified otherwise.

Unless otherwise indicated, all numbers expressing conditions, concentrations, dimensions, and so forth used in the specification and claims are to be understood as being modified in all instances by the term “about.” Accordingly, unless indicated to the contrary, the numerical parameters set forth in the following specification and attached claims are approximations that may vary depending at least upon a specific analytical technique.

The term “comprising,” which is synonymous with “including,” “containing,” or “characterized by” is inclusive or open-ended and does not exclude additional, unrecited elements or method steps. “Comprising” is a term of art used in claim language which means that the named claim elements are essential, but other claim elements may be added and still form a construct within the scope of the claim.

As used herein, the phase “consisting of” excludes any element, step, or ingredient not specified in the claim. When the phrase “consists of” (or variations thereof) appears in a clause of the body of a claim, rather than immediately following the preamble, it limits only the element set forth in that clause; other elements are not excluded from the claim as a whole. As used herein, the phase “consisting essentially of” limits the scope of a claim to the specified elements or method steps, plus those that do not materially affect the basis and novel characteristic(s) of the claimed subject matter.

With respect to the terms “comprising,” “consisting of,” and “consisting essentially of,” where one of these three terms is used herein, the presently disclosed and claimed subject matter may include the use of either of the other two terms. Thus in some embodiments not otherwise explicitly recited, any instance of “comprising” may be replaced by “consisting of” or, alternatively, by “consisting essentially of.”

Devices or system modules that are in at least general communication with each other need not be in continuous communication with each other, unless expressly specified otherwise. In addition, devices or system modules that are in at least general communication with each other may communicate directly or indirectly through one or more intermediaries.

A description of an embodiment with several components in communication with each other does not imply that all such components are required. On the contrary a variety of optional components are described to illustrate the wide variety of possible embodiments of the present invention.

As is well known to those skilled in the art many careful considerations and compromises typically must be made when designing for the optimal manufacture of a commercial implementation any system, and in particular, the embodiments of the present invention. A commercial implementation in accordance with the spirit and teachings of the present invention may configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

It is to be understood that any exact measurements/dimensions or particular construction materials indicated herein are solely provided as examples of suitable configurations and are not intended to be limiting in any way. Depending on the needs of the particular application, those skilled in the art will readily recognize, in light of the following teachings, a multiplicity of suitable alternative implementation details.

Some embodiments of the present invention, and variations thereof, relate to the extraction of moomiyo using an organic solvent. The extracted moomiyo may be used for medical purposes including, but not limited to, joint pain relief, skin care, anti-inflammation, bruise removal, decrease high blood cholesterol and high triglycerides levels, promotion of mental and physical energy, regulation of blood sugar levels, stimulation of the immune system, speeding of the recovery from sickness and exercise and wound healing therapies.

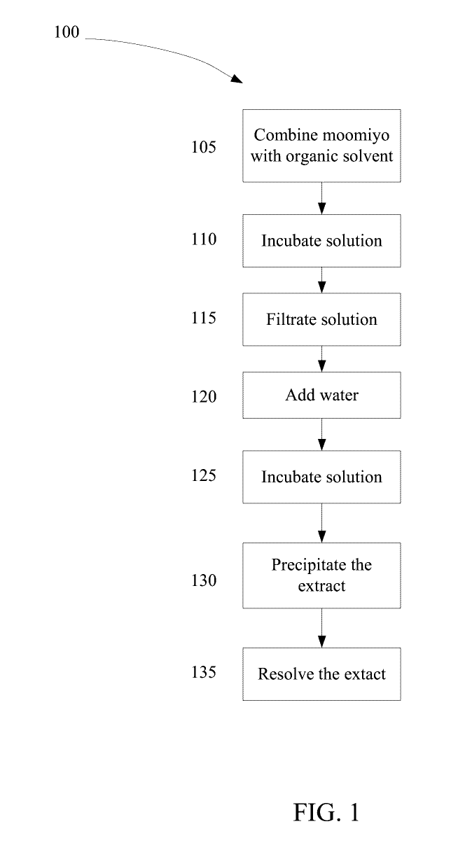

FIG. 1 illustrates a method for moomiyo extraction, in accordance with an embodiment of the present invention. In the present embodiment, moomiyo extraction process starts in a step 105 with moomiyo being combined with an organic solvent. In many embodiments, the moomiyo may be in a powdered form and may be combined with an ethanol solution. In many embodiments, the moomiyo is combined with a 65% ethanol/deionized water solution at a ratio of 1:10 weight/volume (w/v). In other embodiments, the moomiyo extract may be in any form that is suitable for dissolution in the ethanol, the concentration of ethanol may be more or less and the ratio of moomiyo to ethanol solution may be more or less. The concentration of 65% ethanol in water has been chosen because it has maximal extraction activity for a wide range of bio-active compounds.

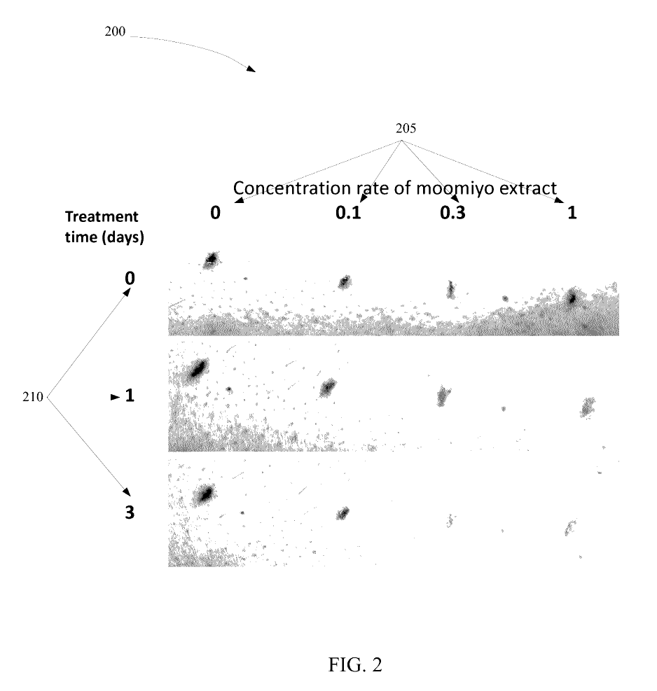

Moomiyo extraction can be performed with ethanol/deionized water at ethanol concentration range 55-85% (preferred range). Moomiyo/extraction solution ratio may vary in range from 1:100 to 1:5. Moomiyo/extraction solution ratio of 1:10 was chosen to get higher concentration of bio-active compound in extract and optimize its yield. The concentration 1:100 is not high enough to detect the wound healing effect (FIG. 2). At concentration 1:5 significant amount of active compound may be lost in the insoluble pellet of moomiyo.

In a step 110 the combined moomiyo and ethanol mixture may be incubated. In many embodiments the mixture may be incubated for approximately one day at room temperature, about 20° C. In other embodiments, the incubation time and temperature may be more or less. Incubation time can be varied from 1 to 10 or more days (preferred range). One day time period was chosen to minimize time of extract preparation. Incubation temperature can be varied in a preferred range from 10 C to 40 C or higher. In most experiments the temperature 20 C has been used usually refereed as “room temperature”. At the end of step 110 the extraction of active compound from row moomiyo is achieved, extract has full biological activity and all following steps are optional. Next steps in the procedure serve for purification of active compound to avoid possible side effects after treatment. In a step 115 the incubated solution may be filtrated to collect a liquid phase of the moomiyo extract. In some embodiments a paper filter may be used to filtrate the solution. In other embodiments other filtration means may be employed. For crude filtration a cotton filter was used sometimes before filtration through the fine filter paper. This pre-filtration significantly decrease the purification time. Next step was filtration through large pore filter paper and on final step fine filtration through Whatman 3MM or similar filter was performed. Basically, for the filtration of moomiyo extract a broad range of filters can be used with comparable results. In some embodiments the collected partially purified crude moomiyo extract 140 may be used for medicinal purposes. In the next optional purification step 120 deionized water may be added to the collected moomiyo extract to a final concentration of about 80%. The preferred range of water concentration may be from 60 to 95% or wider. In other embodiments purified water may be used. Deionized water was prepared by filtration through 2-stage filter with activated carbon layer to trap microorganisms and chemicals and second layer to trap sediments.

In a step 125 the final concentration may be incubated for approximately one day at room temperature, about 20° C. In other embodiments, the incubation time and temperature may be more or less. The preferred incubation time range is from 1 h to several days or longer. The insoluble precipitate formation becomes visible in half an hour after water addition, riches high level 1 day after and does not significantly change for several days later. Time period −1 day was chosen as the most optimal to increase precipitate formation and yield of active compound from one side and decrease the purification time from another side. Incubation temperature may be in range from 10 C to 40 C (preferable range). In most experiments the temperature 20 C has been used as the most usual and comfortable temperature. In a step 130, after incubation the moomiyo extract may be precipitated. In many embodiments the moomiyo extract may be precipitated to form a pellet by centrifuging for about 5 min. at about 3000 rpm and discarding the supernatant. In other embodiments the time and speed may be more or less. Insoluble pellet precipitation can be achieved without centrifugation (by standing tube on the table) but in this way it needs longer time. Centrifugation significantly speeds up the process and saves time. Centrifugation speed and time may be in wide range from 500 rpm-20 min to 10000 rpm-2 min or higher. After removing of the supernatant a pellet has been resolved immediately in the extraction solution. It has not been investigated how pellet can be stored before resolving. In a step 135 the moomiyo extract pellet may be resolved by various means for medicinal purposes. In some embodiment, the moomiyo extract pellet may be resolved in an ethanol solution or an organic oil such as, but not limited to, olive oil. Other organic solvents such as methanol, isopropanol or acetone may be used instead of ethanol. Any vegetable or mineral oil may be used instead of olive oil. In some embodiments moomiyo extract prepared on an oil base may be better for use on large wounded areas or burns. In some other embodiments moomiyo extracts may be added as ingredients into a cream to heal damaged skin.

It may be appreciated to a person with ordinary skill in the art that the above embodiments are not limited to using a set concentration of a single organic solvent and alternative embodiments may use a variety of organic solvents. No restrictions have been found regarding the type of organic solvents that can be used for the extraction. For instance a combination of ethanol with methanol (95:5) or isopropanol (95:5) may be used instead of ethanol.

It may be appreciated to a person with ordinary skill in the art that the step of allowing the solution to sit at room temperature for a day is not limited to a precise time and temperature. Regarding the extraction time it is important to mention that a thin powder of moomiyo has been used in all extractions. That decreased the extraction time significantly compare to intact (stone-like) moomiyo. The minimal time of extraction in this art was 1 day. No difference in bio-activity was detected when extraction time was increased from 1 to 7 days or longer. Similarly no difference in bio-activity was detected at different extraction temperature in range 10 C-40 C.

It may be apparent to a person with ordinary skill in the art that there may be many ways or alternatives to filtering and centrifuging a solution. Alternative embodiments of the invention may use alternative filtering and centrifuging methods. The solubility of moomiyo in the ethanol is about 5% which means that a bulk of insoluble material presents in the extraction solution and must be separated from the active compound. Various procedures may be used for the separation. In the present art a filtration through several filters was used for this purpose. Other methods of separation such as centrifugation can be applied too.

FIG. 2 depicts the effect of the ethanol extracted moomiyo on wound healing in a dose-dependent manner, in accordance with an embodiment of the present invention. In the present embodiment, moomiyo powder was incubated in 65% ethanol in a 1 to 20 weight-to-volume ratio (w/v) for one day at room temperature. The test skin was sterilized with 95% alcohol and wounded with abrasive paper. Dilutions of the moomiyo extract with concentrations 205 of 0, 0.1, 0.3, and 1.0 corresponding 0%, 10%, 30% and 100% of moomiyo extract in 65% ethanol respectively were prepared in 65% ethanol and 0.3 milliliters were applied on the fabric pads of sterile bandages to cover the wounds. Every day the bandages were removed, the wounds cleaned with soap water, and sterilized with 95% ethanol before being covered with fresh bandages containing the moomiyo extract. In the present embodiment, pictures of the wounds were taken on days 210 0, 1 and 3. Wounds with increased concentrations of moomiyo extract appear to have stronger wound healing activity, which may be an indication of higher bioactive compounds in the solution applied to the wound.

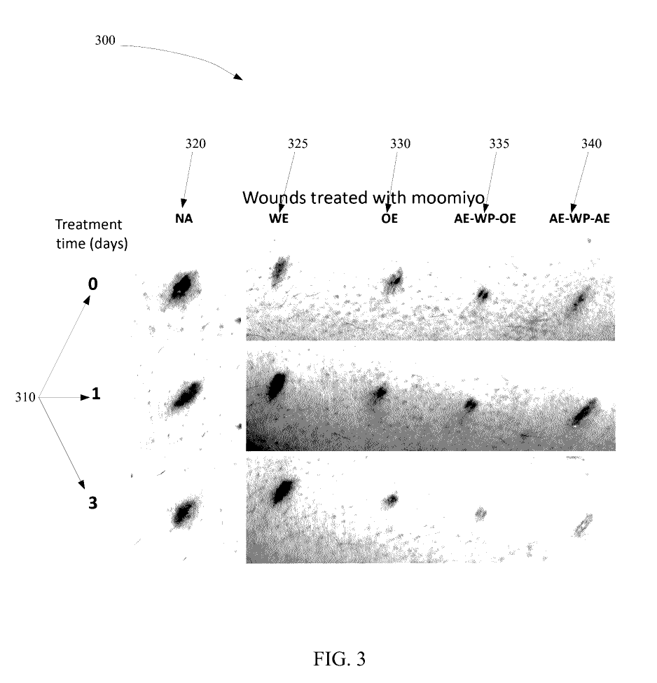

FIG. 3 depicts the effect of different moomiyo extracts on wound healing, in accordance with an embodiment of the present invention. In the present embodiment, water extract (WE) 325 of moomiyo was prepared by incubation of moomiyo powder with sterile deionized water in 1:20 (w/v) ratio for 1 day at room temperature and following filtration. Oil extract (OE) 330 of row moomiyo was prepared similarly by incubating with pure olive oil. Ethanol extract of moomiyo was diluted (1:3) with deionized water and incubated 1 day at room temperature. Supernatant was removed and pellet dissolved in the original volume of 65% ethanol (AE-WP-AE) 340 or the same volume of pure olive oil (AE-WP-OE) 335. Wound treatment was performed as described in above for FIG. 2. Wounds treated with 65% ethanol were used as a negative control (NA) 320. Top, middle and bottom panels represent 0, 1 and 3 days of treatment respectively 310. Water extract of row moomiyo 325 showed no detectable wound healing activity over days 310. Precipitate formed in alcohol extract of moomiyo after 1:3 dilution with water demonstrated strong wound healing activity after dissolving in 65% ethanol 340 as well as in pure olive oil 335. Oil extract 330 of moomiyo showed a weak or no healing activity.

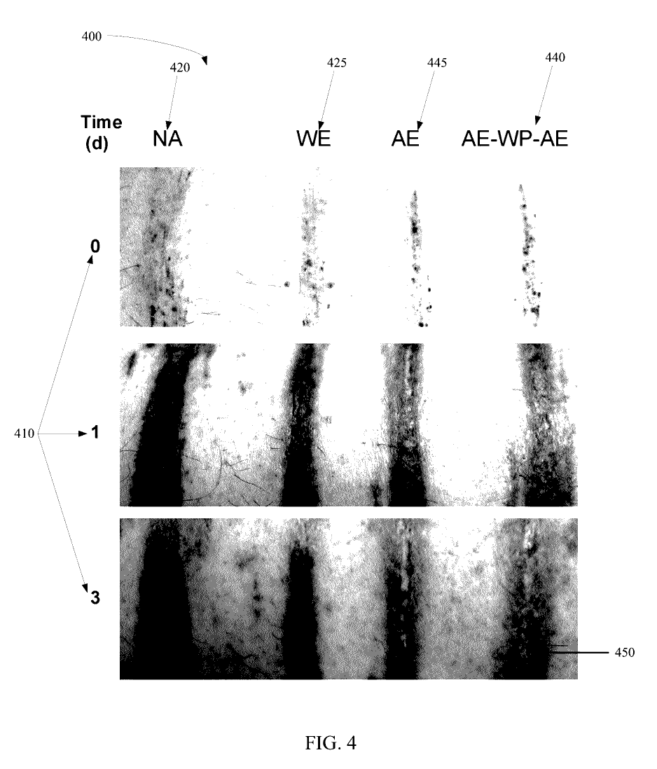

FIG. 4 depicts the effect of alcohol extracted moomiyo on granulation tissue formation, in accordance with an embodiment of the present invention. In the present embodiment, alcohol (AE) and water (WE) extracts of moomiyo as well as water insoluble fraction from alcohol extract of moomiyo dissolved in 65% ethanol were prepared as described above for FIG. 2 and FIG. 3. Skin on the leg was treated with abrasive paper to make larger wounds than in the previous experiments. After sterilization with 95% alcohol each wound was covered with Strong Strips sterile adhesive bandage (2 in×4 in) with fabric pad soaked with 0.6 ml of 65% ethanol (NA) 420, water extract of moomiyo (WE) 425, 65% ethanol extract of moomiyo (AE) 445 and water precipitated fraction from alcohol extract of moomiyo dissolved in 65% ethanol (AE-WP-AE) 440. Wounded skin was treated and photographed as above. Top, middle and bottom panels represent 0, 1 and 3 days of treatment respectively 410. Arrow 450 shows a granulation tissue formation in the wounded area. Granulation tissue formation can be seen in moomiyo extract treated wounds 425, 445 on the first day of the treatment.

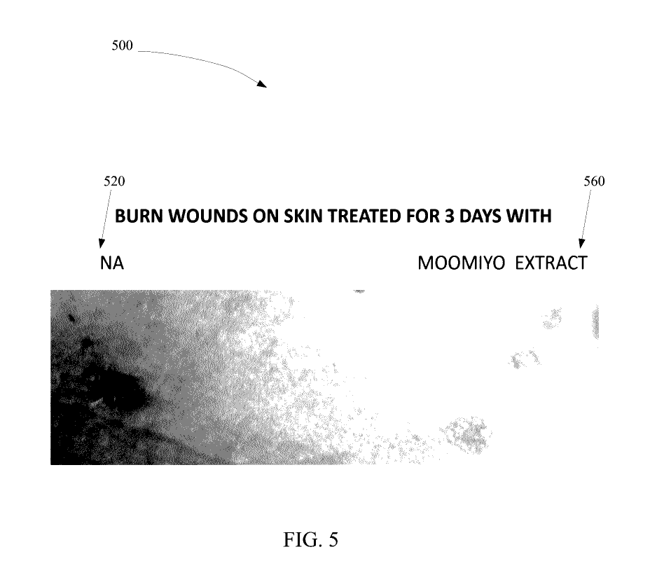

FIG. 5 depicts the effect of moomiyo cream on wound healing, in accordance with an embodiment of the present invention. In the present embodiment, cosmetic cream was made by mixing of 10 ml of 65% ethanol extract of moomiyo (1g moomiyo powder per 10 ml of ethanol) with 90 ml of a cosmetic cream base. An amount of 0.3 ml of the cream was applied daily on the burn skin wounds formed in the result of cosmetic benign epithelial tumor-papilloma removal. Untreated wound served as a negative control (NA) 520. Moomiyo containing cream 560 significantly improved burn skin wounds healing.

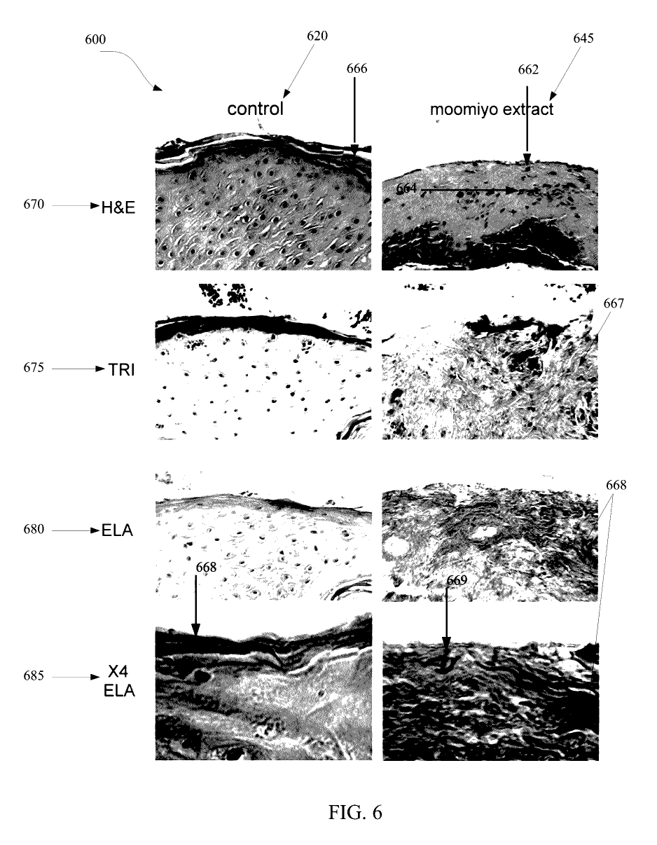

FIG. 6 is a histology analysis of wounded skin, in accordance with an embodiment of the present invention. In the present embodiment, skin on the leg was wounded with abrasive paper as described in FIG. 4. The wounds were treated with ethanol extract of moomiyo 645 or 65% ethanol (control) 620 as before. Four days later small pieces of tissue were taken from the wounded areas. Tissue was fixed in 10% buffered formamide for 18 h, processed on automated tissue processor, embedded in paraffin and cut on microtome by 5 um thick sections. Slides were stained with hematoxylin and eosin stain (H&E) 670, Masson's trichrome stain (TRI) 675 and Verhoeffs elastic stain ELA 680 and ×4 ELA 685. Hematoxylin and eosin stain demonstrates that wound treated with moomiyo extract 645 formed a thick layer of granulation tissue containing an extracellular matrix with embedded fibroblast cells 662 and a number of small capillary blood vessels 664. In the control 620 tissue a layer of granulation tissue with embedded fibroblasts was very thin and sometimes hard to find 666. In trichrome stained tissue collagen fibers of extracellular matrix are colored blue, nuclei are black and red blood cells, muscles and cytoplasmic components are red. In wound treated with moomiyo extract 645 an abundant amount of thick collagen fibers 667 stained blue can be seen. No such staining can be detected in the control tissue. In the case of elastic stain 680 and 685 two components of extracellular matrix—elastic fibers and collagen are getting black and red colors respectively. Wound tissue treated with moomiyo extract 645 shows strong collagen-related red staining 668. In control tissue the week red staining can be seen only at higher magnification (×4) in thin surface layer. Thin elastic fibers stained black can be detected at high magnification in the extracellular matrix 669. They were spread all over thick layer of extracellular matrix in the wound treated with moomiyo extract 645. In control 620 some elastic fiber can be seen only on the surface of wound tissue together with collagen 669.

Extracting moomiyo with a mixture of organic solvents and water may yield high concentrations of bioactive compounds. The extraction with a mixture of organic solvents and water is a relatively simple, cheap, and efficient method compared to current methods of moomiyo extraction. The extracted moomiyo may also be used in medical therapies. Experimental trials show a strong correlation between extracted moomiyo application on wounds and wound healing activity.

In some embodiments, wound healing activity in moomiyo may be capable of activating fibroblasts migration, extracellular matrix synthesis and new blood vessels formation in the wound area. Some embodiments may provide joint pain relive activity with moomiyo extract added into cream or taken per-orally to significantly reduce joint pain in individuals with osteoarthritis, rheumatoid arthritis, lower back pain and etc. Some embodiments may provide aged skin regenerating activity with moomiyo extract added into cream may dramatically improve aged skin appearance and show wrinkle removing activity. Some embodiments may provide anti-inflammation activity with moomiyo extract to reduce inflammation process in the tissue. Some embodiments may provide bruise removing activity with a cream containing moomiyo extract to inhibit bruise formation.

Those skilled in the art will readily recognize, in light of and in accordance with the teachings of the present invention, that any of the foregoing steps may be suitably replaced, reordered, removed and additional steps may be inserted depending upon the needs of the particular application. Moreover, the prescribed method steps of the foregoing embodiments may be implemented using any physical and/or hardware system that those skilled in the art will readily know is suitable in light of the foregoing teachings. For any method steps described in the present application that can be carried out on a computing machine, a typical computer system can, when appropriately configured or designed, serve as a computer system in which those aspects of the invention may be embodied.

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

Having fully described at least one embodiment of the present invention, other equivalent or alternative methods of implementing the extraction of moomiyo from an organic solvent and water mixture according to the present invention will be apparent to those skilled in the art. Various aspects of the invention have been described above by way of illustration, and the specific embodiments disclosed are not intended to limit the invention to the particular forms disclosed. The particular implementation of the extraction of moomiyo from an organic solvent and water mixture may vary depending upon the particular context or application. By way of example, and not limitation, extraction of moomiyo from an organic solvent and water mixture described in the foregoing were principally directed to medical therapy applications such as promoting wound healing; however, similar techniques may instead be applied to the extraction of any or all bioactive compounds from solutions which implementations of the present invention are contemplated as within the scope of the present invention. The invention is thus to cover all modifications, equivalents, and alternatives falling within the spirit and scope of the following claims. It is to be further understood that not all of the disclosed embodiments in the foregoing specification will necessarily satisfy or achieve each of the objects, advantages, or improvements described in the foregoing specification.

Claim elements and steps herein may have been numbered and/or lettered solely as an aid in readability and understanding. Any such numbering and lettering in itself is not intended to and should not be taken to indicate the ordering of elements and/or steps in the claims.

The corresponding structures, materials, acts, and equivalents of all means or step plus function elements in the claims below are intended to include any structure, material, or act for performing the function in combination with other claimed elements as specifically claimed.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The Abstract is provided to comply with 37 C.F.R. Section 1.72(b) requiring an abstract that will allow the reader to ascertain the nature and gist of the technical disclosure. It is submitted with the understanding that it will not be used to limit or interpret the scope or meaning of the claims. The following claims are hereby incorporated into the detailed description, with each claim standing on its own as a separate embodiment.

What is claimed is:

1. A method for extraction of moomiyo from a multi-component, naturally-occurring mineral substance generally found in Asian mountain ranges at elevations approximately between 1000 and 5000 meters for medicinal applications, the method comprising the steps of:
combining said multi-component, naturally-occurring mineral substance with an organic solvent mixed with deionized water, wherein said organic solvent comprising a predetermined ratio of ethanol mixed with said deionized water, in which said predetermined ratio of ethanol with said deionized water is configured for substantial extraction of bio-active compounds from said mineral substance;
incubating said combined multi-component, naturally-occurring mineral substance and organic solvent for a predetermined period of time at a predetermined temperature range;
filtering said incubated multi-component, naturally-occurring mineral substance and organic solvent, in which said filtering step comprising collecting a liquid phase of a moomiyo extract from said multi-component, naturally-occurring mineral substance;
adding a predetermined amount of water to said moomiyo extract, in which said water comprises at least one of filtered water and deionized water substantially free of at least one of microorganism, chemical and sediment;
incubating said moomiyo extract for a predetermined amount of time at a predetermined temperature range;
precipitating said moomiyo extract, in which said precipitating step further comprising the steps of discarding a supernatant and forming at least one of moomiyo pellet, moomiyo cream and moomiyo powder;
resolving said moomiyo pellet for said medicinal applications.

2. The method of claim 1, in which said medicinal applications comprising wound healing applications.

3. The method of claim 2, wherein said at least one of moomiyo pellet, moomiyo cream and moomiyo powder is resolved in at least one of ethanol solution, organic solvent and organic oil for said wound healing applications.

4. The method of claim 3, in which said wound healing applications comprising at least one of skin regenerating activity, wrinkle removing activity and bruise removing activity.

5. The method of claim 1, in which said medicinal applications comprising at least one of reducing joint pain, anti-inflammation activity, decrease high blood cholesterol and high triglycerides levels, promotion of mental and physical energy, regulation of blood sugar levels, stimulation of the immune system, and speeding of the recovery from sickness.

6. A system for extraction of moomiyo from a multi-component, naturally-occurring mineral substance for medicinal applications, the system comprising:
means for combining said multi-component, naturally-occurring mineral substance with an organic solvent mixed with water, in which said organic solvent comprising a predetermined ratio of ethanol with said water, wherein said predetermined ratio of ethanol with said water is configured for maximal extraction of bio-active compounds from said mineral substance;
means for incubating said combined multi-component, naturally-occurring mineral substance and organic solvent for a predetermined period of time at a predetermined temperature range;
means for filtering said incubated multi-component, naturally-occurring mineral substance and organic solvent, wherein said filtering step is configured to collect a liquid phase of a moomiyo extract from said multi-component, naturally-occurring mineral substance;
means for adding a predetermined amount of water to said moomiyo extract, in which said water comprises filtered water substantially free of at least one of microorganism, chemical and sediment;
means for incubating said moomiyo extract for a predetermined amount of time at a predetermined temperature range;
means for precipitating said moomiyo extract, in which said precipitating step further comprising the steps of forming a pellet and discarding a supernatant;
means for resolving said moomiyo extract for said medicinal applications.

7. The system of claim 6, in which said medicinal applications comprising wound healing applications.

8. The system of claim 7, wherein said at least one of moomiyo pellet, moomiyo cream and moomiyo powder is resolved in at least one of ethanol solution, organic solvent and organic oil for said wound healing applications.

9. The system of claim 8, in which said wound healing applications comprising at least one of skin regenerating activity, wrinkle removing activity and bruise removing activity.

10. The system of claim 9, in which said medicinal applications comprising at least one of reducing joint pain, anti-inflammation activity, decrease high blood cholesterol and high triglycerides levels, promotion of mental and physical energy, regulation of blood sugar levels, stimulation of the immune system, and speeding of the recovery from sickness.

11. The system of claim 11, in which said mineral substance is generally found in Asian mountain ranges at elevations approximately between 1000 and 5000 meters.

12. A method for extraction of moomiyo from a multi-component, naturally-occurring mineral substance for medicinal applications, the method comprising the steps of:
combining said multi-component, naturally-occurring mineral substance with an organic solvent mixed with water, wherein a predetermined ratio of organic solvent is mixed with water being configured to substantially extract bio-active compounds from said mineral substance;
incubating said combined multi-component, naturally-occurring mineral substance and organic solvent for a predetermined period of time at a predetermined temperature range;
filtering said incubated multi-component, naturally-occurring mineral substance and organic solvent, wherein said filtering step is configured to collect a liquid phase of a moomiyo extract from said mineral substance;
adding a predetermined amount of water to said moomiyo extract, in which said water comprises water substantially free of at least one of microorganism, chemical and sediment;
incubating said moomiyo extract for a predetermined amount of time at a predetermined temperature range;
precipitating said moomiyo extract, in which said precipitating step further comprising the step of forming at least one of moomiyo pellet, moomiyo cream and moomiyo powder;
resolving said moomiyo pellet for said medicinal applications.

13. The method of claim 12, in which said medicinal applications comprising wound healing applications.

14. The method of claim 2, wherein said at least one of moomiyo pellet, moomiyo cream and moomiyo powder is resolved in at least one of ethanol solution, organic solvent and organic oil for said wound healing applications.

15. The method of claim 3, in which said wound healing applications comprising at least one of skin regenerating activity, wrinkle removing activity and bruise removing activity.

16. The method of claim 1, in which said medicinal applications comprising at least one of reducing joint pain, anti-inflammation activity, decrease high blood cholesterol and high triglycerides levels, promotion of mental and physical energy, regulation of blood sugar levels, stimulation of the immune system, and speeding of the recovery from sickness.

17. The method of claim 11, in which said mineral substance generally found in Asian mountain ranges at elevations approximately between 1000 and 5000 meters.

18. The method of claim 11, in which solvent solution comprising at least one of ethanol solution, methanol solution, isopropanol solution and acetone solution.

19. The method of claim 11, in which solvent solution comprising, at a predetermined ratio, at least one of ethanol solution combined with methanol solution, ethanol solution combined with isopropanol solution and ethanol solution combined with acetone solution.

20. The method of claim 11, in which said water comprising at least one of deionized water and filtered water, and in which said predetermined period of time comprising at least 1 day.
